(19) United States
(12) Patent Application Publication
(10) Pub. No.: US 2023/0034506 A1
(43) Pub. Date: Feb. 2, 2023

(54) A DEVICE FOR THE INSPECTION OF STEEL WIRE ROPES AND A PROCEDURE FOR THE USE THEREOF

(71) Applicant: Mike Pfennig, Leipzig (DE)

(72) Inventors: Falk Sonntag, Leipzig (DE); Christian Hofmann, Leipzig (DE)

(21) Appl. No.: 17/620,841

(22) PCT Filed: Jun. 8, 2020
(86) PCT No.: PCT/EP2020/025266

§ 371 Date: Dec. 20, 2021

(30) Foreign Application Priority Data
Jun. 18, 2019 (DE) 102019004240.9

(51) Int. Cl. G01N 27/83; G01L 1/16; G01L 1/22; G01N 33/20

(52) U.S. Cl. CPC G01N 27/83; G01L 1/16; G01L 1/22; G01N 33/20

(57) ABSTRACT

A device and procedures perform an inspection of steel wire ropes and steel chains. The device is formed from a minimum of two contactless housing segments that are positioned around a test specimen and are equidistant connected when open. The housing segments have an internal normal surface that is radial and center-oriented when closed. The housing segments also form a supporting housing for all of the other components, and being connected on one of its longitudinal sides as a minimum. The connection being realized by moving connecting pieces for defining the clearance, rotation axis and the opening angles of each of the housing segments and which have a suitable closing mechanism positioned on a freely definable pair of longitudinal sides of the housing segment for the purpose of locking when in a closed condition and in order to apply a required pressing force.

The invention concerns a device for the inspection of steel wire ropes and a procedure for the use thereof, especially for the inspection of suspended steel wire ropes or chains.

The inspection device for the non-destructive inspection of steel wire ropes should serve to provide a location-independent inspection of steel wire ropes with various diameters that are to be used in an installed, freely suspended, or section by section tensioned condition.

The carrier cables that are especially used for elevator systems are deemed to be safety-relevant operating materials and only have a restricted useful life. This is the reason why the condition of the cables is to be inspected at regular intervals.

Such an inspection of lifting gear with conventional wire ropes is normally carried out in the form of an expert carrying out a visual inspection during recurring or regular inspections of the entire installation. It is normally the case here that wire breakages are determined and counted. A random testing of the cable diameter is also manually carried out.

An advanced inspection of the mounted steel wire rope is only possible after the rope has been removed so that a manual inspection can be carried out, or it has to be moved along by a person so that it can be manually and externally inspected.

The removal of the rope results in downtime and therefore unproductive times for the user of work platforms for example.

When moving along the rope, either the rope that is to be inspected is to be used although its condition is not known, or additional ropes have to be installed, whereby this also results in downtimes.

One example of a solution for inspections that go further than external inspections is presented in EP 0286712.

This describes a device for the inspection of ferromagnetic steel wire ropes, by which the lifting mechanism rope that is to be inspected, is driven through a field of permanent magnets, resulting in the stray fields being detected, whereby at least an induction coil that is to preferably encompass the rope and the measuring device is downstream from an electronic evaluation unit, it being intended in the invention that the stray fields are detected by means of differential measuring coils and that at least one additional induction coil with a downstream integrator emits a signal that reflects the cross-section of the rope, this being registered with an opto-electronic or electric scanning of the lifting mechanising rope that is to be inspected, the determined metallic cross-section and the sum of the stray fields, together with a path marker and a time marker.

EP 2 383 566 A1 describes a procedure for a computer aided optical inspection of a rope that comprises:—the provision of an image data set showing at least a section of the rope using a camera for example;—the provision of target values for a visual longitudinal elongation of wires relative to the visual longitudinal elongation of the wire in the image data set;—determination of a visual longitudinal elongation of the wires in the image data set, whereby the determination comprises an adaptation of a presumed longitudinal elongation to the image data set;—determination of at least one quality value by means of a quality standard as a function for the determination of a certain visual longitudinal elongation of the wires and the target values of the visual longitudinal elongation of the wires;—discrimination of visual positions within the image data set of the rope, with which at least one quality value exceeds or falls short of a predetermined allocated quality value;—the provision of the discriminated visual positions and a related system, together with a computer program.

Another procedure for the inspection of the suspension means of lifting mechanisms, especially ropes in elevator systems is described in EP 1 914 186 31 A1 with the following steps:—the permanent provision of an evaluation unit on a lifting mechanism,—temporary provision of at least one sensor device relating to a sensor for the determination of sensor signals as a basis for the inspection of the discard criteria of the suspension means on the lifting mechanism,—In operative connection of the permanently provided evaluation unit with the temporary provided sensor device for the evaluation of the determining sensor signals for the provision of information regarding the discard criteria as regards the suspension means.

It is the task of the invention to recommend a solution that remedies the disadvantages of the known state of the art and by means of which, steel wire ropes or chains can be inspected when fastened, without it being necessary for a person to move along them and inspect them manually, whereby the time that is required for the inspection is shortened and an improved proof of the inspection and improved documentation is provided.

An additional manual evaluation of the measured data is then no longer necessary.

This task is solved by the device that is the subject of the invention and the procedure for its use which is described in more detail on the basis of the illustrations and execution examples.

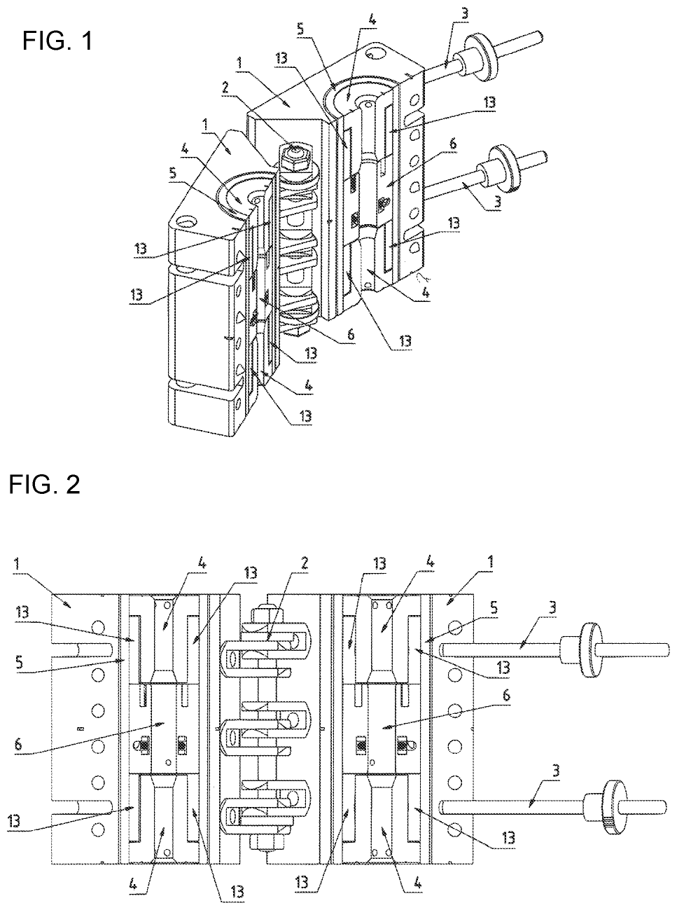

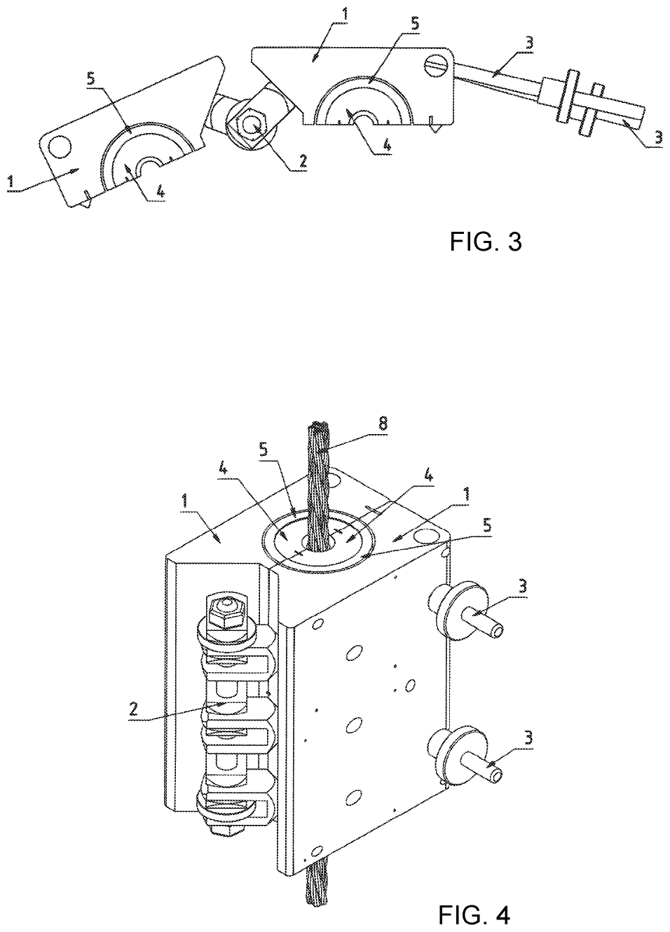

FIGS. 1 to 3 hereby depict the device that is the subject of the invention when open,

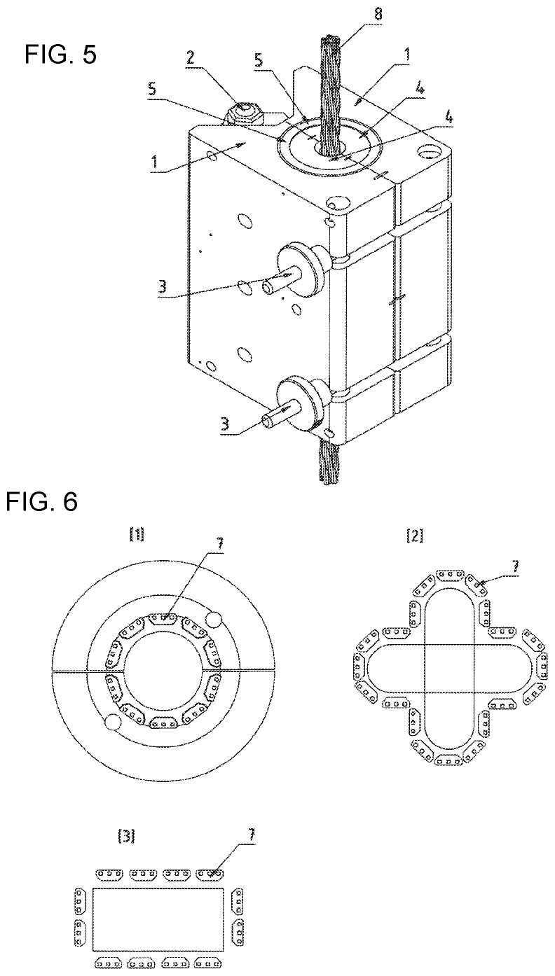

FIGS. 4 and 5 depict the device that is the subject of the invention when closed, and

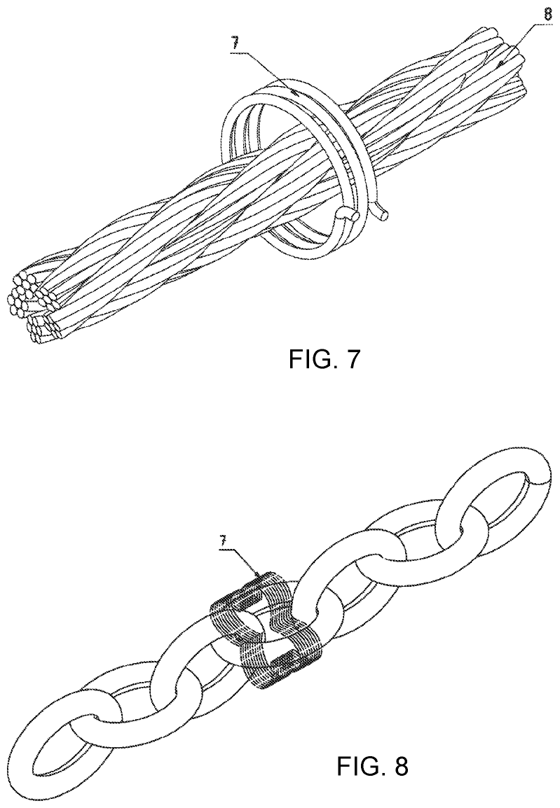

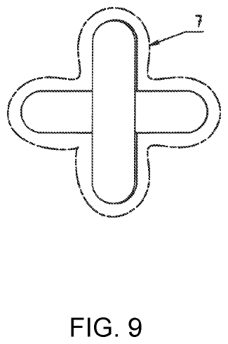

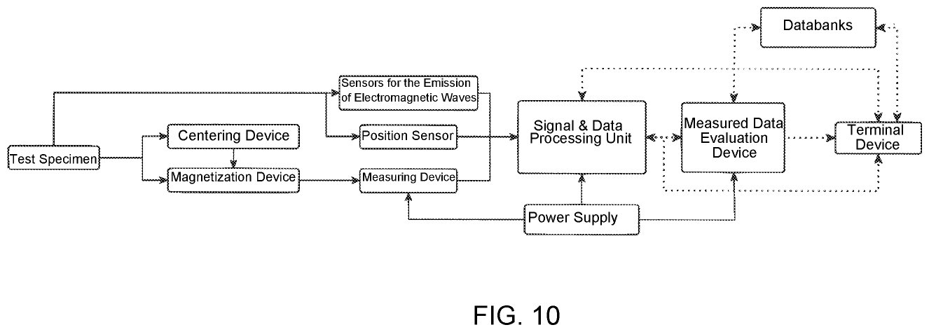

FIGS. 6 to 9 depict variants of the sensor and FIG. 10 depicts a signal flow diagram.

The main constituent parts of the sensor device, the signal and data processing unit 11 and the measured data evaluation unit 12 are of a modular design so that they can be adapted and combined for special applications.

This advantageous design enables the device that is the subject of the invention to be put to diverse uses such as when carrying out installation work on crane systems (e.g. gantry cranes) and as a direct connection with the crane control unit.

The device that is the subject of the inventions can also be installed at the outlet of winding machines for the manufacturing of steel wire ropes as a quality assurance method during the manufacturing process or in order to monitor ropes or chains on installation machines or crane systems.

The main field of application of the device that is the subject of the invention is certain to be the inspection of ropes and chains in the conveyance of people and loads (e.g. passenger elevators) and (interior) access systems in wind power plants.

The device that is the subject of the invention is formed by at least two housing segments 1, that are positioned around a test specimen 8 in a contactless form and are connected at an equidistance and are internally normal surface and centre-oriented when closed, such forming a supporting housing for all the other components.

These housing segments 1 are connected with each other by at least one of their longitudinal sides by means of mobile connecting pieces 2 for the defining of the clearance, rotation axis and the opening angles of each of the housing segments 1.

A suitable closing mechanism 3 is positioned on a freely definable pair of longitudinal sides of the housing segment 1 for the purpose of locking when in a closed condition and in order to apply the required pressing force.

The device that is the subject of the invention also has a multi-piece centering device 4 brought together by the housing segments 1 when in a closed condition, for the purpose of applying the relative force when decentralising the test specimen 8 from the guide axis of the centring device 4.

The device that is the subject of the invention also has a multi-piece magnetization device 5 that is brought together by the housing segments 1 when in a closed condition, this being for the generation of a magnetic field that flows through the test specimen 8 in segments, this field being homogenous and magnetically saturating, with 4 collinear longitudinal axes to the guide axis of the centring device 4.

The magnetisation device 5 has a measuring device 6 inside it with sensor elements 7 that are segmentable, automatic, electric contactable, and relate to the circumference of the test specimen 8 in sections, these also being equidistant arranged, these elements serving the measuring of the magnetic fields created by the test specimen 8 and the transferring of the measured signals to a signal and data processing unit 11.

Other features of the device that is the subject of the invention are
a minimum of one multi-piece measuring device 6 that is mounted in any position whatsoever on the housing and contains sensors for the emission of electromagnetic waves 10 from the test specimen 8 and the transmission of the measuring signal to a signal and data processing unit 11;
a minimum of one position sensor 9 that is integrated in the device and serves the determination of the length of the test specimen 8, the position of the defects and the path that has been travelled by the device if applicable, in order to determine the translatory movement parameters of the test specimen 8, such as the relative speed between the test specimen 8 and the device and the time required for the transmission of the signals to a signal and data processing unit;
a minimum of one signal and data processing unit 11 that is either integrated in the device, or is cableless and portable, the unit being for the preparing of the signals and the generation, processing, storage, and transmission of the measured values;
a minimum of one data transfer capable measured data evaluation unit 12 that is either integrated or segregated and serves the pattern recognition, fault classification and data correlation for the issuing of a diagnosis report and the transmission of all the data to a terminal device 14;
a minimum of one electrical power supply 15 for the supplying of power to the measuring devices 6, the signal and data processing unit 11, and the measured data evaluation unit 12.

There now follows a more detailed description of the elements of the device that is the subject of the invention, on the basis of design examples:

The housing comprises two symmetrically identical segments 1 with a guide groove and markings for the exact positioning of the segments in relation to each other. The guide groove can be supplemented by mechanical positioning devices.

In another design form, the housing can comprise more than two components.

Connection 2 of the symmetrically identical housing segments 1 is formed by three fork joints with a joint rotation axis and fixation nuts by way of example. Numerous connecting elements 2 can be mounted for any number of housing segments 1. The fork joints that are depicted by way of example can be replaced by other rigid or flexible connecting elements 2.

Double transverse groove bolts with a threaded rod and knurled nuts serve as a locking mechanism 3.

The required pressure torque can be achieved by using mechanical (e.g. tensioning mechanisms, screw closures, pneumatic toggle clamps), hydraulic (e.g. hydraulic clamps) or electromechanical (a servomotor, a brushless dc, or a dc motor) locking mechanisms.

A special design is that the solution that is the subject of the invention can include a monitoring and controlling of the pressure force in the form of strain measuring strips that are inserted in the locking mechanism 3 or in the housing.

The centring device 4 should preferably comprise a rotationally symmetric, two-component guiding sleeve that exists double.

The centring device 4 can be of a flexible and variable design that enables diverse rope cross-sections and rope geometries to be centred, depending on the number of wires or strands, imbalance, and the winding direction. The geometries of the guiding sleeves or other centring devices 4 can hereby be adapted to the form of the test specimen 8 as required. The geometry of the guiding sleeves can therefore also be designed for the centring of chains.

The centring device 4 can be replaced by a pressure mechanism that can be integrated in a running gear if necessary. Mechanical sensors such as strain measuring strips or piezoelectric elements can be mounted in a running gear if necessary for the monitoring and controlling of the required pressure force.

The consideration behind the inventions also includes a multi-part designing of the centring device 4.

In another design, the centring device 4 can be contactless and electromagnetically controlled.

An alternative to saving, the centring device 4 can be realized with the assistance of the measured data evaluation unit 12, as this makes an exact depiction of the test specimen 8 possible, irrespective of its relative position within the device.

In a special design, the magnetization device 5 is formed of diametral magnetized, equidistant, rotationally symmetric arranged ring segment magnets 13 with an iron counterplate, with eight being on each half of the sensor, with four forming a common inner north pole and four forming a common inner south pole.

The magnetization device 5 also has a ferromagnetic iron counterplate for the concentration of the magnetic lines of force in the outer area.

The ring segment magnets 13 can be replaced with other forms and types of magnets or by electromagnets.

Additional sensors 7 can be used to monitor and control an electromagnetic magnetization device 6, in order to ensure an adequately high and homogenous magnetic flow.

The magnetization device 5 can be designed so that it can be adapted to various test specimen geometries.

Another possible design of the magnetisation device 5 is realised by a coil arrangement such as a Helmholtz coil for example.

The measuring device 6 is preferably to be in two symmetrical parts and form a holder for equidistantly arranged sensors 7, such as Hall sensors, it also being possible that it can comprise induction loop segments that encompass the test specimen 8. The measuring device 6 includes electric (signal) cable feedthroughs. In addition to the holder, the measuring device 6 also protects the sensor elements 7 against mechanical damage.

As an alternative, the measuring device 6 can also be mounted outside but in close proximity to the magnetization device 5.

The positioning of the sensor elements 7 that are included in the measuring device 6, can be automatically controlled by a mechanism and the output signals can therefore be adapted to the geometry of the test specimen.

The measuring device 6 can be of a multi-piece design.

In another design, the measuring device 6 converts analogue sensor signals into digital measured values and transmitted between the housing segments 1.

The measuring device 6 can be adapted to various test specimen geometries. An example for this is depicted in FIG. 6.

Another advantageous design of the device is one that contains infrared sensors for the scanning of the surface profile of the steel wire ropes or chains that are to be inspected, in order to obtain an improved differentiation between various types of faults such as corrosion or surface soiling.

A position sensor 9 can also be in the form of an incremental shaft encoder or as an optical sensor (e.g. laser).

A signal and data processing unit 11 can be divided on housing segment 1 or it can be positioned centrally. The signal and data processing unit 11 is able to record, store, and transmit all the measured data simultaneously at all times of measurement.

A measured data evaluation unit 12 can classify faults automatically by performing a feature extraction from the measured data. The collected data are entered in a database 16 that is either inside or outside the device, so as to ensure a traceability of the measurements and render temporal changes of the test specimen 8 between different measurements discernible.

It is also possible to enter target values and the specific characteristics of the test specimen 8 in the database 16 in advance, so that an immediate pattern recognition and fault classification can be ensured.

The evaluated data can be output for further processing. The measured data can hereby be transferred to a terminal device 14 that is able to visualise or store data in a graphical, figurative, tabular form or in the form of a measured data file. As an alternative, a visualisation possibility (e.g. a screen) can be mounted on the device. The transfer of the measured data to the terminal device 14 can be carried out by means of a wireless data transfer (e.g. WLAN).

The measured values evaluation unit 12 does not necessarily have to be connected to the signal and data processing unit 11.

A signal processing algorithm that is included in the measured data evaluation unit 12 means that the centring device 4 is not required.

The procedure for the use of the device that is the subject of the invention shall now be explained on the basis of the signal flow diagram FIG. 10:
manual linking of the centring device 4 of the device that is the subject of the invention, to the test specimen 8;
enclosure of the test specimen 8 and locking of housing segments 1 by applying the required force of attraction against the internal repulsive force by means of the locking device 2, closing of all electric contacts;
a magnetically saturated magnetic flowing through the test specimen 8 by the magnetization device 5 and the creation of magnetic axial and radial fields conform with the geometric profile and the permeability of the test specimen 8;
induction of a relative movement between the device and the test specimen 8 along the longitudinal axis of the centring device 4;
transfer of the magnetic profile of the test specimen 8 via the sensors 7 of the measuring device 6 to analogue measured signals;
automatic calibration and adjustment of the signal and data processing unit (11);
simultaneous implementation and storage of the analogue measuring signals from all sensors 7 in a digital data flow by the signal and data processing unit 11;
early detection of faults by the signal and data processing unit 11 in real-time and the transferring of the fault location to a device, preferably a mobile terminal device 14;
synchronous calculation of the geometric profile of the test specimen 8 by the measured data evaluation unit 12 for the measuring process on the basis of the magnetic profile;
sample recognition and classification of fault locations and generation of an overall view by the measured data evaluation unit 12;
automatic generation of a diagnosis on the basis of a comparison being made between the overall view and the target parameters of the test specimen 8 by the measured data evaluation unit 12;
transfer of the specific diagnosis reports to a database 16 in order to detect any temporal changes and creation of a prognosis;
storage and output of a test report to terminal device 14.

Additional advantageous designs of the solution that is the subject of the invention are as follows, whereby this is non-conclusive:
a possible combination of a drive unit for the movement of the rope;
an adaptation of the component geometry of ring segment magnets 13, iron counterplate, holder and the guiding sleeves can be adapted for the measuring of similar test specimen geometries such as chains, Chinese fingers, and various cross-sections;
an adaptation of the data processing unit to the measurement of similar test specimen geometries such as chains, Chinese fingers, etc.
an upgrading of the device with sensors for the measuring of electromagnetic wave emissions 10 (e.g. infrared) for an improved classification of the type of fault;
an upgrading of the device with optical sensors (e.g. a camera) in order to visualise the faults;
replacing the transverse groove bolts with an electromechanical closure unit;
an upgrade for an automatic and dynamic calibration of the measuring device 6 and the signal and data processing unit 11.

It is also possible to initialise the measured value recording by means of the incremental shaft encoder of the position sensor 9 so as to ensure a conformity between the measurement point and the corresponding measured value.

An immediate checking of the test specimen identity is possible should the target values for the diameter, imbalance, number of wires/strands, the winding direction [right/left], etc. have been entered in the used database 16.

An inspection of other rope elements such as thimbles, is also possible using an optical imaging process.

Typical applications for the device that is the subject of the invention and the 19 proceedings for its use are:

The device that is the subject of the intervention can be permanently installed by combining the sensor 7 and the signal and data processing unit 11, and thereby ensure a permanent monitoring of the installation that is to be inspected. The feature of the device that suffices for this is the early fault detection in real-time that is ensured by omitting the advanced evaluation of the measured data without the measured data evaluation unit 12.

Specific examples for this are:

The device can be used in a mobile form after it has been coupled with a drive system that should preferably have a self-sufficient source of energy.

This enables the following typical applications to be stated:
the direct environment of the test specimen can be inspected for specimens that come into contact with the test specimen 8 so that they can disturb the normal operation;
inspection of tensioning systems that are used for fixing in place in high installations (e.g. radio towers);
advance inspection of supporting and securing ropes of external movement installations (e.g. rotor blade or façade access equipment);
inspection of bridge tensioning ropes (e.g. from pylon bridges);
in the event of severe damage being caused that meets the adequate discard criteria for test specimen 8, the device can complete the inspection prematurely with the corresponding diagnosis.
The adaptability of the measuring device 6, the magnetization device 5, and the signal and data processing unit 11 regarding the test specimen geometry, any form of load handling devices (e.g. lifting gear, chain hoists) can be inspected as long as they are fitted with ferromagnetic power transmission elements. These include any configurations with round link chains, roller chains, stud chains, etc.

### LIST OF REFERENCE SIGNS

1. Housing segment

2. Connecting piece

3. Closing mechanism

4. Centring device

5. Magnetization device

6. Measuring device

7. Sensor element

8. Test specimen

9. Position sensor

10. Sensors for an emission of electromagnetic waves

11. Signal and data processing unit

12. Measured data evaluation unit

13. Ring segment magnet

14. Terminal device

15. Energy supply

16. Database

1-24. (canceled).

25. A device for inspecting test specimens being steel wire ropes and steel chains, the device comprising:
at least two housing segments disposed around a test specimen in an equidistant form when open and when closed, said at least two housing segments having a radial, center-oriented internal surface normal and forming a supporting housing for other components;
moving connecting pieces for connecting said at least two housing segments and disposed on a longitudinal side of each of said at least two housing segments in order to define a clearance, a rotation axis and an opening angle of each of said at least two housing segments;
a locking mechanism disposed on a defined pair of longitudinal sides of said at least two housing segments for locking in a closed position;
a multi-part centering device being brought together when said at least two housing segments are closed by said locking mechanism and applying a reactive force when decentralizing the test specimen from a guide axis of said multi-part centering device;
a multi-part magnetization device being positioned together by said at least two housing segments when closed and serving to generate a magnetic field that homogenously flows through the test specimen section by section and magnetically saturates the test specimen, said multi-part magnetization device having a collinear longitudinal axis leading to the guide axis of said centering device;
a signal and data processing unit;
a measuring device with segmentable, automatically contactable sensor elements that relate to a circumference of the test specimen and positioned in an equidistant form and are for measuring magnetic fields being generated locally and for transferring measured signals to said signal and data processing unit;
at least one position sensor for determining a length of the test specimen, a position of faults and a distance travelled by the device to determine translatory movement parameters of the test specimen including a relative speed between the test specimen and the device and to transmit position sensor signals to said signal and data processing unit;
said signal and data processing unit being either integrated in the device or is in a cableless and portable form and serves for processing signals in addition to generating, processing, storing and transmitting measured values;
a terminal device;
at least one integrated or segregated measured data evaluation unit for pattern recognition, fault classification and data correlation for issuing a diagnosis report and a transmission of all data to said terminal device; and
at least one electrical power supply for a supply of power to said measuring device, said signal and data processing unit and said measured data evaluation unit.

26. The device according to claim 25, further comprising strain measuring strips being inserted in said locking mechanism or said supporting housing in order to monitor and control a pressure force.

27. The device according to claim 25, wherein said multi-part centering device for an inspection of the steel chains includes a rotationally symmetric, two-piece guiding sleeve that exists double.

28. The device according to claim 27, wherein said multi-part centering device is in a flexible and variable design and conforms with geometries of said rotationally symmetric, two-piece guiding sleeve or other centering devices, in addition to a geometry of the test specimen.

29. The device according to claim 27, wherein a geometry of said rotationally symmetric, two-piece guiding sleeve is suitable for a centering of the steel chains.

30. The device according to claim 25, further comprising a controller; and
wherein said segmentable, automatically contactable sensor elements used in said measuring device, being an electromagnetic magnetization device, in order to ensure a high and homogeneous magnetic flow, this being monitored and controlled by said controller.

31. The device according to claim 30, wherein said multi-part magnetization device has a geometric configuration that conforms with corresponding test specimen geometries.

32. The device according to claim 25, further comprising a mechanism, a positioning of said segmentable, automatically contactable sensor elements in said measuring device is automatically controlled by said mechanism.

33. The device according to claim 25, wherein a geometric design of said measuring device conforms with various test specimen geometries.

34. The device according to claim 25, further comprising infrared sensors for scanning a surface profile of the steel wire ropes or the steel chains that are to be inspected, in order to provide an improved differentiation between different forms of faults.

35. The device according to claim 25, wherein said at least one position sensor functions as an optical sensor.

36. The device according to claim 25, further comprising a drive unit for moving a steel wire rope.

37. The device according to claim 25, further comprising ring segment magnets, iron counterplates, and retaining and guiding sleeves for measuring of similar test specimen geometries that conform with geometries of steel chains, Chinese fingers, and varying cross-sections.

38. The device according to claim 25, wherein said signal and data processing unit is adapted for measuring similar test specimen geometries.

39. The device according to claim 25, further comprising sensors for measuring the electromagnetic wave emissions in order to improve a classification of types of the faults.

40. The device according to claim 25, further comprising optical sensors for a visualization of the faults.

41. A method for inspecting test specimens being steel wire ropes and steel chains using a device, which comprises the steps of:
moving a centering device of the device towards a test specimen;
encompassing the test specimen by locking housing segments of the device by applying a required force of attraction against an internal repulsive force by means of a locking device of the device, for closing all electric contacts;
generating a magnetically saturating flow through the test specimen via a magnetization device of the device and generating magnetic axial and radial fields which conform with geometric profiles and a permeability of the test specimen;
inducing a relative movement between the device and the test specimen along a longitudinal axis of the centering device;
transferring a magnetic profile of the test specimen via analogue measured signals by sensors of a measuring device of the device;
automatically calibrating and adjusting a signal and data processing unit;
simultaneously implementing and storing of the analogue measured signals from all the sensors in a digital data flow by the signal and data processing unit;
detecting faults via the signal and data processing unit and a transferring of a position of the faults to a terminal device;
performing a measuring process in a form of a synchronous calculation of a geometric profile of the test specimen by a measured data evaluation unit of the device on a basis of the magnetic profile;
performing a pattern recognition and classification of fault locations and generating an overall view by the measured data evaluation unit;
automatically generating a diagnosis on a basis of a comparison being made between the overall view and target parameters of the test specimen by the measured data evaluation unit;
transferring specific diagnosis reports to a database in order to detect any temporal changes and create a prognosis; and
storing and issuing a test report to a terminal device of the device.

42. The method according to claim 41, which further comprises using the measured data evaluation unit to automatically classify the faults on a basis of features extraction from the measured data with the measured data being automatically entered into the database that is inside or outside the device, in order to ensure a traceability of measurements and render the temporal changes of the test specimen between different measurements discernible.

43. The method according to claim 42, which further comprises entering advance information regarding target values and specific characters of the test specimen in the database in order to ensure an immediate pattern recognition and fault classification.

44. The method according to claim 42, which further comprises outputting evaluated data for further processing so that the measured data can be transferred to the terminal device that is able to visualize or store data in a graphical, figurative, tabular form or in a form of a measured values file.

45. The method according to claim 42, wherein in order to transfer the measured data to the terminal device, using a visualization possibility including a screen that is mounted on a device control system and a wireless data transfer system being a wireless local area network.

46. The method according to claim 42, which further comprises carrying out an initialization of a recording of the measured values by means of an incremental shaft encoder of the position sensor in order to obtain an agreement between a measured site and a measured value that is as high as possible.

47. The method according to claim 43, which further comprises entering target values for a diameter, an imbalance, a number of wires/strands, and a winding direction in the database so that an immediate identification of the test specimen can be carried out.

48. The method according to claim 42, which further comprises using an optical imaging process in order to inspect other rope elements including thimbles.
